(19) United States
(12) Patent Application Publication
(10) Pub. No.: US 2024/0272093 A1
(43) Pub. Date: Aug. 15, 2024

(54) SYSTEM AND METHOD FOR PROVIDING A DIGITALLY SWITCHABLE X-RAY SOURCES

(71) Applicant: NANO-X IMAGING LTD., NEVE-ILAN (IL)

(72) Inventors: Ukyo JEONG, MORGAN HILL, CA (US); Amir BEN SHALOM, MODI?IN (IL)

(21) Appl. No.: 18/018,911

(22) PCT Filed: Aug. 10, 2022
(86) PCT No.: PCT/IB2022/057477

§ 371 Date: Jan. 31, 2023

Related U.S. Application Data
(60) Provisional application No. 17/419,725, filed on Jun. 30, 2021.
(60) Provisional application No. 62/810,410, filed on Feb. 26, 2019.
(60) Provisional application No. 62/786,593, filed on Dec. 31, 2018.

(30) Foreign Application Priority Data
Aug. 9, 2021 (KR) 10-2021-0104789

(51) Int. Cl. G01N 23/04; A61B 6/00; A61B 6/40; G01N 23/083

(52) U.S. Cl. CPC G01N 23/04; A61B 6/405; A61B 6/482; A61B 6/5235; G01N 23/083; G01N 2223/204; G01N 2223/401

(57) ABSTRACT

Material of an object is detected by an x-ray imaging device capturing a sample set of x-ray images at various anode voltages comparing the x-ray images and using differences between the x-ray images of the sample set to determine material of an imaged object. A composite image is synthesized of the object from the x-ray images representing different materials constructing the object

### FIELD OF THE DISCLOSURE

The disclosure herein relates to systems and methods for determining material composition of an object using x-ray imaging. In particular, the disclosure relates to an apparatus and method configured to determine the material of an object based on images of an object obtained according to a set of a plurality of x-ray images obtained by performing x-ray imaging of the object using anode voltages of various sizes.

### BACKGROUND

X-ray imaging devices include an X-ray source (X-ray tube) that generates X-rays and a detector (detector) that detects the X-rays transmitted through the subject and converts them into an image. The X-ray source is generated by accelerating the electrons emitted in the vacuum tube towards an anode target and the accelerated electrons striking the Anode electrode. An electric field-emitting X-ray source, which is a type of X-ray source, is a cathode electrode disposed in a vacuum tube, an electron emitter installed on the cathode electrode, and it includes a gate electrode and an anode electrode installed adjacent to the electron emitter, and is configured to emit electrons by an electric field formed between the gate electrode and the electron emitter. When an electron beam emitted from the electron emitter proceeds through such a hole, electrons are accelerated by an electric field formed between the anode electrode and the cathode electrode, and the X-ray is emitted by hitting the X-ray target installed on the anode side by an accelerated electron beam.

Conventional x-ray imaging devices are used to determine whether organs in the body are abnormal from images obtained based on X-ray imaging of a subject or to detect dangerous substances inside a travel carrier bag. On the other hand, materials having different material properties are known to each have different radiation densities for the X-ray spectrum. For example, x-rays may be obtained for objects with different materials by setting different voltages of the anode electrodes, such as 20 kV, 40 KV, and 80 kV, and X-ray images of objects having different materials on several occasions. In this example, objects having materials such as paper or plastic are also brightly transmitted in x-rays with low energy and high energy, so that there is no difference in contrast in the images, but objects with materials such as metals have a clear light and dark difference according to the difference in energy, so that the difference in contrast is prominent.

The present invention is intended to solve the aforementioned problem, and it is a technical task to provide a device and a method configured to determine the material of an object based on images of an object obtained according to a plurality of X-ray images by setting the anode voltage differently.

The technical challenges that the present invention intends to achieve are not limited to the technical tasks described above, and other technical challenges of the present invention can be derived from the following description. Thus, there is a need for controllable x-ray sources with fast response times. The invention described herein addresses the above-described needs.

### SUMMARY OF THE EMBODIMENTS

As a technical means for solving the above-described technical problems, embodiments according to the first aspect of the present invention provide a method for determining the material of an object through an X-ray imaging device and a communication connection of the server. The method involves the X-ray imaging apparatus receiving a sample set of a plurality of X-ray images obtained by performing X-ray imaging of the subject using anode voltages of various sizes, the server receiving transmission from the X-ray imaging device. The server performs a step of determining the material of objects included in the sample set of X-ray images based on the difference in the sample X-ray images according to the anode voltage size, and further of synthesizing the sample set of X-ray images by the anode voltage size and generating a final X-ray image for the subject based on the material information of the objects included in the sample X-ray images by the anode voltage size determined in the synthesized image.

In addition, embodiments according to another aspect of the present invention provide a material determination system of an object using X-ray imaging. The present system includes a communication module for transmitting and receiving information with an X-ray imaging device, a memory for storing an X-ray program, and a processor for executing the X-ray program. The processor executes the X-ray program, wherein the X-ray imaging apparatus receives a sample set of a plurality of X-ray images obtained by performing X-ray imaging of the subject using anode voltages of various sizes through the communication module, and determines the material of the objects included in the subject X-ray images by the anode voltage size based on the difference in the subject X-ray images by the anode voltage size. In addition, the subject set of X-ray images by the anode voltage size are synthesized and the material information of the objects included in the sample set of X-ray images by the anode voltage size determined in the synthesized image is configured to generate a final X-ray image for the subject.

In addition, an embodiment according to another aspect of the present invention is an X-ray imaging device for obtaining a plurality of anode voltage size subject X-ray images by performing X-ray images of the subject by the anode voltage size and receiving the X-ray images by the anode voltage size from the X-ray imaging apparatus and receiving the subject X-ray images by the anode voltage size, and determining the material of the objects included in the sample X-ray images by the anode voltage size, In addition, the material determination system of the object using X-ray imaging, comprising a server configured to generate a final X-ray image for the subject by the object reflecting the material information of the objects included in the object X-ray images by the anode voltage size determined by the synthesized image and determined in the synthesized image.

According to the present invention, by setting various levels of the anode voltage, an X-ray imageic image captured at each anode voltage can be collected, and a reconstructed subject X-ray image image can be obtained by combining the same.

In addition, according to the present invention, various anode voltage can be selected so as to collect X-ray images of the subject at the variety of anode voltage levels, and based on this, the material of the subject and the objects present inside the subject may be determined.

The effects of the present invention are not limited to the above-described effects, but include all effects understood from the following description.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the various selected embodiments may be put into practice. In the accompanying drawings:

FIG. 1 is a block diagram representing selected elements of an embodiment of a switchable x-ray source;

Fig, 2 schematically represents a possible electron emitting construct for use in embodiments of the switchable x-ray source;

FIG. 3 is a block diagram representing of another embodiments of a switchable x-ray source incorporating an synchronized optical imager;

FIG. 4 illustrates possible signal profiles of a shutter signal and a gate signal and the resulting imaging rate acquired by an optical imager imaging an irradiated scintillator;

FIGS. 5A-C schematically represent another embodiment of the x-ray source incorporating an synchronized optical imager;

FIG. 6 is a graph illustrating how tube current varies with Filament current for a thermal emission x-ray tube;

FIGS. 7A-E indicate various timing examples of synchronization signals;

FIG. 8A is a diagram illustrating a configuration of an object material determination system using X-ray imaging according to an embodiment of the present invention;

FIG. 8B is a block diagram illustrating the detailed configuration of the server shown in FIG. 8A;

FIG. 8C is a diagram illustrating the operation of an object material determination system using X-ray imaging shown in FIG. 8A;

FIG. 8D is a diagram illustrating an experimental example using an object material determination system using X-ray imaging shown in FIG. 8A;

FIG. 8E is a flowchart illustrating selected steps of a method for determining material using X-ray imaging according to another embodiment of the present invention; and

FIGS. 8F and 8G are diagrams illustrating the detailed steps for some steps of the object material determination method using X-ray imaging shown in FIG. 8G.

### DETAILED DESCRIPTION

Aspects of the present disclosure relate to systems and methods for determining material composition of an object using x-ray imaging. In particular, the disclosure relates to an apparatus and method configured to determine the material of an object based on images of an object obtained according to a set of a plurality of x-ray images obtained by performing x-ray imaging of the object using anode voltages of various sizes.

Such systems may utilize digitally switchable x-ray sources such as the controlled stroboscopic x-ray sources are introduced which may enable regular periodic high frequency x-ray pulses which can be synchronized with other periodic signals described in U.S. patent application Ser. No. 17/419,725 the contents of which are incorporated herein by reference in its entirety.

In various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally, or alternatively, the data processor may access a non-volatile storage, for example, a magnetic hard-disk, flash-drive, removable media or the like, for storing instructions and/or data.

It is particularly noted that the systems and methods of the disclosure herein may not be limited in their application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting.

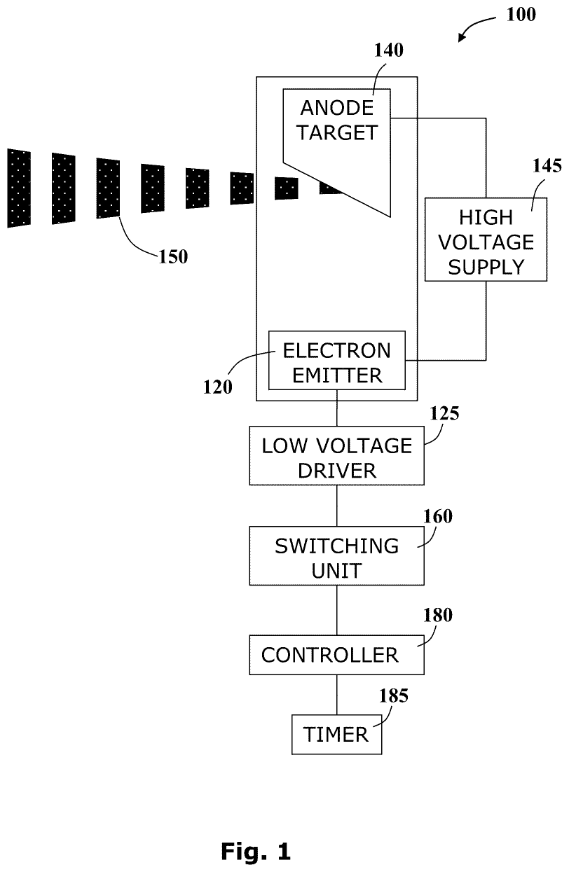

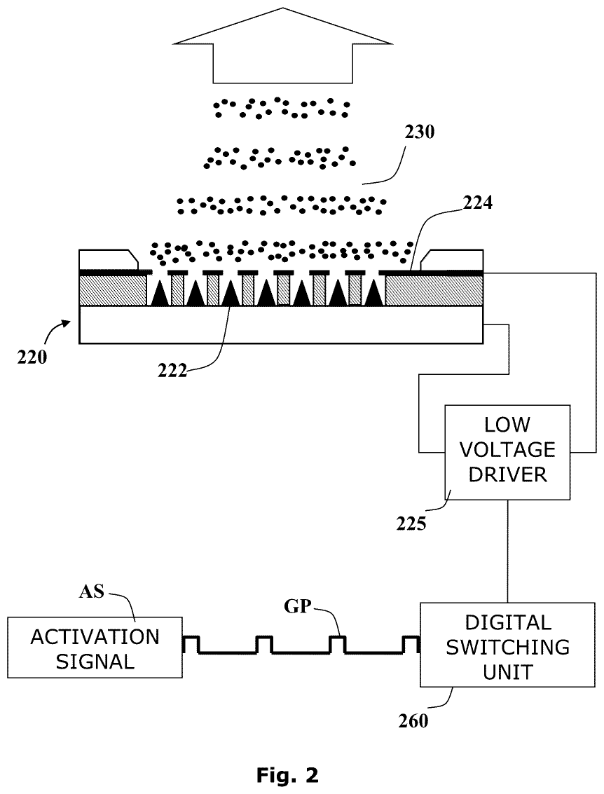

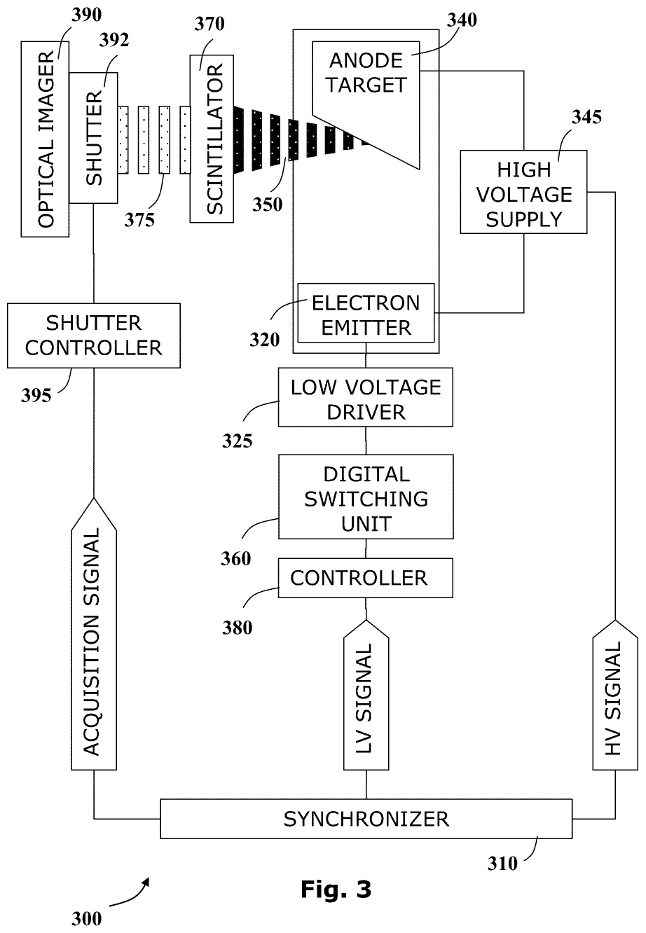

FIG. 1 is a block diagram representing selected elements of an embodiment of a switchable x-ray source 100. The digitally switchable x-ray emission system 100 includes an electron emitter 120, an anode target 140, a high voltage supply 145, a low voltage driver 125, a switching unit 160 a controller 180 and a timer. 185

The electron emitter 120 may be a cold cathode such as a low voltage activated field emission type electron emitting construct configured and operable to release electrons when stimulated by a low voltage. Accordingly, the low voltage driver 125 may include a low voltage driving circuit for activating the electron emitting construct;

The anode target 140 may comprise a metallic target selected such that x-rays 150 are generated when it is bombarded by accelerated electrons from the electron emitter 120. The anode 140 may be constructed of molybdenum, rhodium, tungsten, or the like or combinations thereof.

The high voltage supply 145 wired between said electron emitting construct 120 and the anode 140 is provided for establishing an electron accelerating potential between said electron emitting construct 120 and the anode 140.

It is a particular feature of the digitally switchable x-ray emission system 100 that the digital switching unit 160 is provided to selectively connect and disconnect the low voltage driving circuit 125 thereby selectively activating and deactivating the electron emitting construct 120. Accordingly, emission of the electrons may be controlled by the digital switching system 160.

When the emitting construct 120 is activated electrons are accelerated towards said anode target 140 and a pulse of x-rays 150 is generated. As a result, x-ray emission from the anode 140 may be controlled digitally by the switching unit 160.

The controller 180 may be provided to generate an activation signal which can control the switching rate of the digital switching unit 160. It is particularly noted that in contrast to high voltage switching systems, because the activation signal is a low voltage signal, the response time of the electron emitter is much shorter than the response time of switching the high voltage accelerating potential.

As a result of the reduced response time of the low voltage switching unit, a timer 185 may be provided to generate a fixed clock signal and a high frequency activation signal may be provided consisting of a series of short duration gate pulses at regular intervals.

Referring now to FIG. 2, which schematically represents a possible electron emitting construct 120 for use in embodiments of the switchable x-ray source. A field emission type electron source 122 may be electrically connected to a driving circuit 225 via a signal line and further electrically connected to a gate electrode 224. The coordinated electrical activation of the driving circuit and the gate electrode 224 connected to a field emission type electron source 222 results in its activation, i.e., electron emission. The field emission type electron source 222 performs the electron emission 230 by an electric field formed between the field emission type electron source 222 and the gate electrode 224.

The field emission type electron source 222 may be, e.g., a Spindt type electron source, a carbon nanotube (CNT) type electron source, a metal-insulator-metal (MIM) type electron source or a metal-insulator-semiconductor (MIS) type electron source. In a preferred embodiment, the electron source 222 may be a Spindt type electron source.

The activation signal AS may comprise a series of gate pulses GS generated at a regular intervals Δt and having a fixed gate-pulse duration δt1. Accordingly, the electron emission 230 may follow a similar regular pattern of emission.

With reference to the block diagram of Fig, 3 which represents another embodiment of a switchable x-ray source 300 incorporating an synchronized optical imager 390.

The x-rays 350 emitted by the x-ray source 340 may be directed towards a scintillator 370 such that the scintillator 370 fluoresces when a pulse of x-rays 350 is incident thereupon. The optical imager 390 is configured and operable to detect florescence 375 from the scintillator 370 when its shutter 392 is open.

A shutter controller 395 is provided to trigger the shutter 392 of the optical imager when a shutter pulse is received.

It is noted that a synchronizer 310 may be provided to synchronize a shutter signal with the electron emission activation signal to further control the imaging duration of the system. Accordingly, the synchronizer may be operable to coordinate a high voltage (HV) signal, a low voltage (LV) signal and an acquisition signal.

The high voltage signal may be a function over time determining the characteristics of the high voltage amplitude of the electron accelerating potential produced by the high voltage supply 345. The signal profile of the HV signal may be controlled by the synchronizer 310 and coordinated with the LV signal and the acquisition signal to control the imaging rate of an x-ray device 300.

The low voltage signal may be a function over time determining the characteristics of the switching rate determined by the controller 380 of the digital switching unit 360. The digital switching unit 360 accordingly may activate the low voltage driver 325 for producing the low voltage activation potential provided to the electron emitting construct 320. The LV signal profile may be controlled by the synchronizer 310 and coordinated with the HV signal and the acquisition signal to control the imaging rate of an x-ray device.

The acquisition signal may be a function over time determining the sampling rate of the optical imager 390. Accordingly, by controlling the acquisition signal and coordinating it with the HV signal and the LV signal the synchronizer 310 may control the imaging rate of an x-ray device 300.

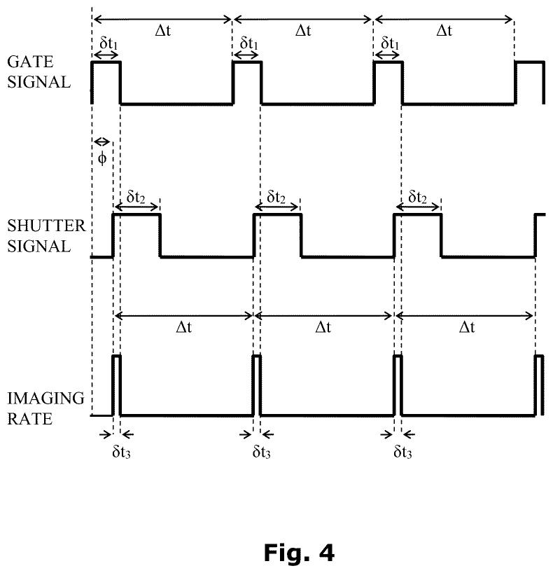

FIG. 4 illustrates possible signal profiles of a shutter signal and a gate signal and the resulting imaging rate acquired by an optical imager imaging an irradiated scintillator. The Gate Signal comprises a series of gate pulses generated at a regular intervals Δt and having a fixed gate-pulse duration δt1. The Shutter Signal has the same frequency and consists of a phase shifted series of trigger pulses generated at the same regular intervals Δt and having a fixed shutter-pulse duration δt2. The Gate Signal may be synchronized to the shutter signal such that the start of each shutter-pulse of the shutter signal is offset from the start of each gate-pulse by a phase shift ϕ. Accordingly, the imaging rate is determined by the frequency (same Δt intervals) but the effective exposure time during which the optical imager accumulates optical stimulation is determined by the overlap between the two signals δt3.

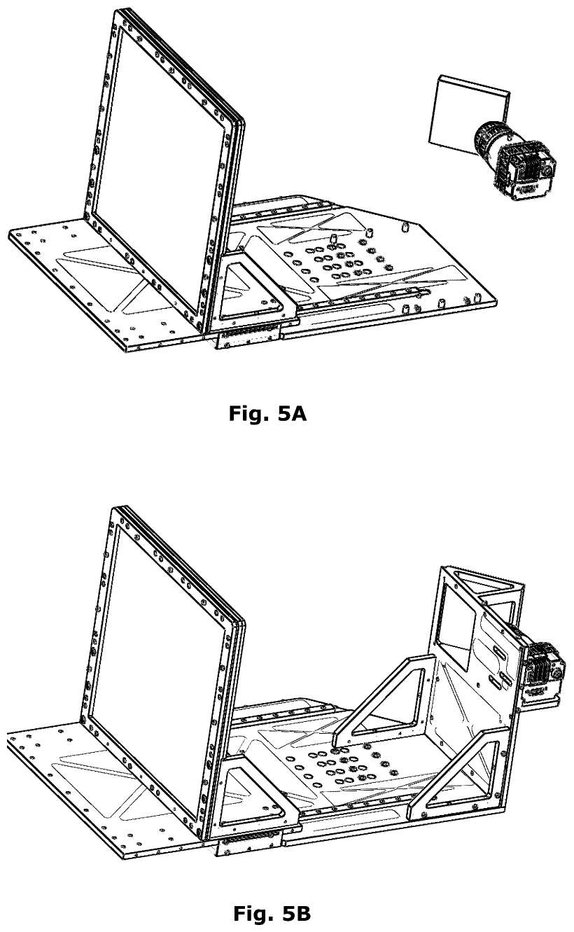

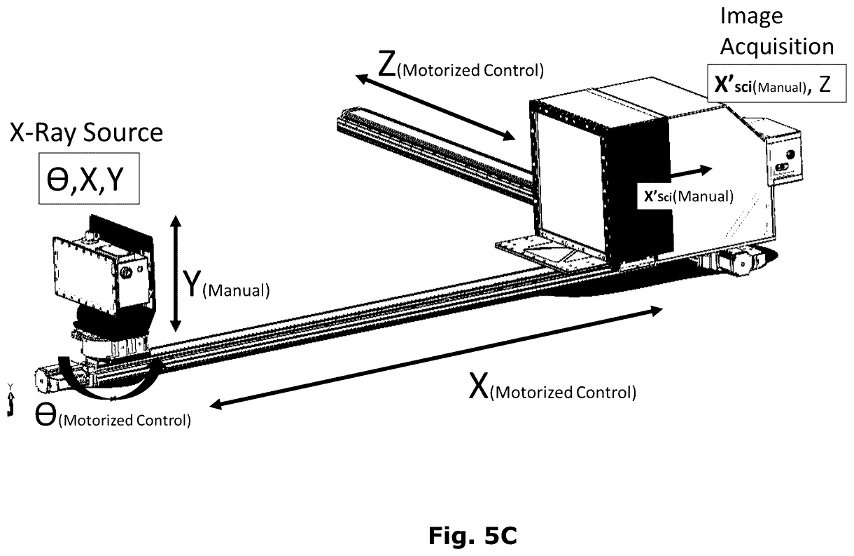

FIGS. 5A-C schematically represent another embodiment of the x-ray source incorporating a synchronized optical imager. FIG. 5A shows an image acquisition unit including a scintillator target, and optical imager configured such that the scintillator target forms an angle of forty-five degrees to both the optical imager and the. FIG. 5B shows a housing configured to secure the scintillator target and the optical imager at the desired angle. FIG. 5C shows how the image acquisition may be configured to receive x-rays from an x-ray source.

It is noted that a field emission (FE) cathode by contrast to standard hot filament x-ray sources have a gate electrode which is operable at relatively low voltages of only tens of volts This gate electrode, practically “ejects” the electrons from the cathode and control the amount of x-ray radiation.

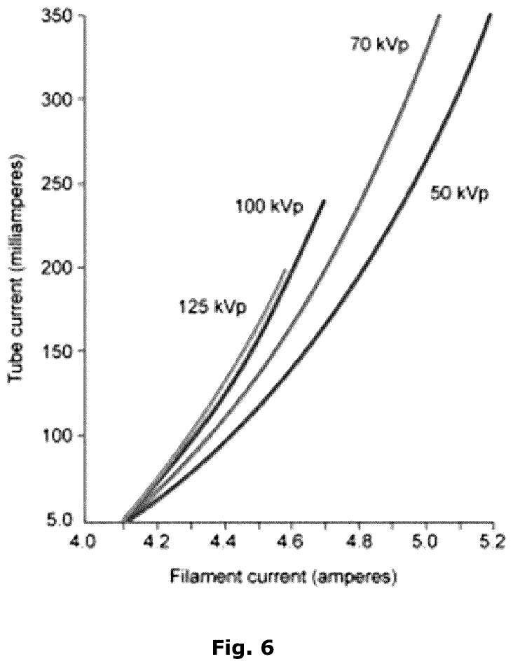

This enables the x-ray power (mA tube current) to be controlled separately from the accelerating voltage (KVp). In thermal emission, the tube current depends upon the high voltage potential difference and on the filament temperature (see example plot in FIG. 6). Such a current can stabilized/changed very slowly in the second scale. In field emission sources, tube current can be set by the gate voltage level that can change rapidly on a microsecond scale.

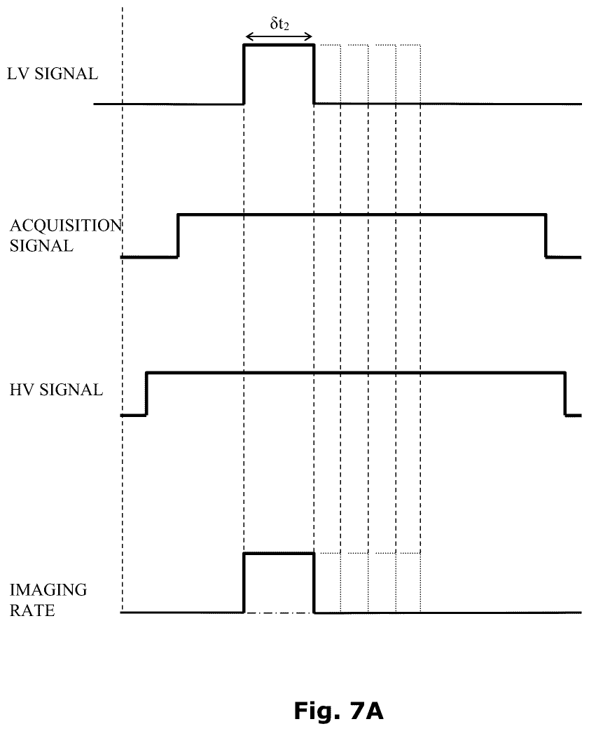

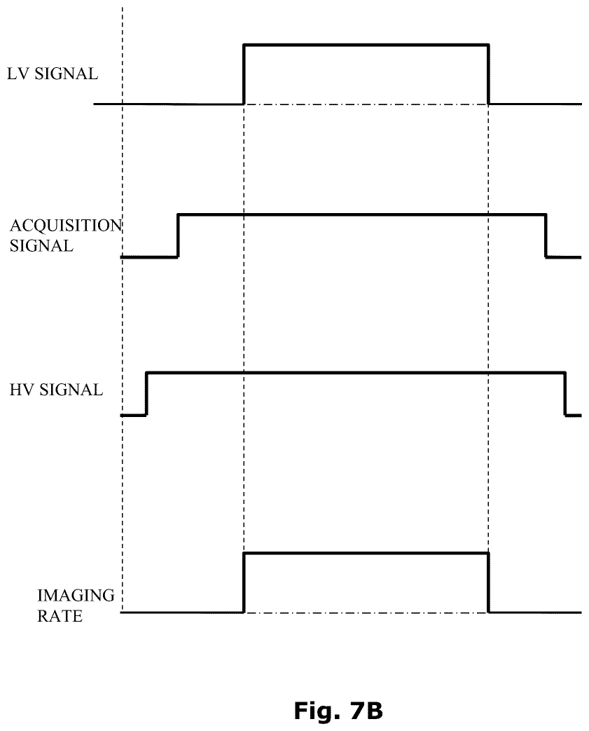

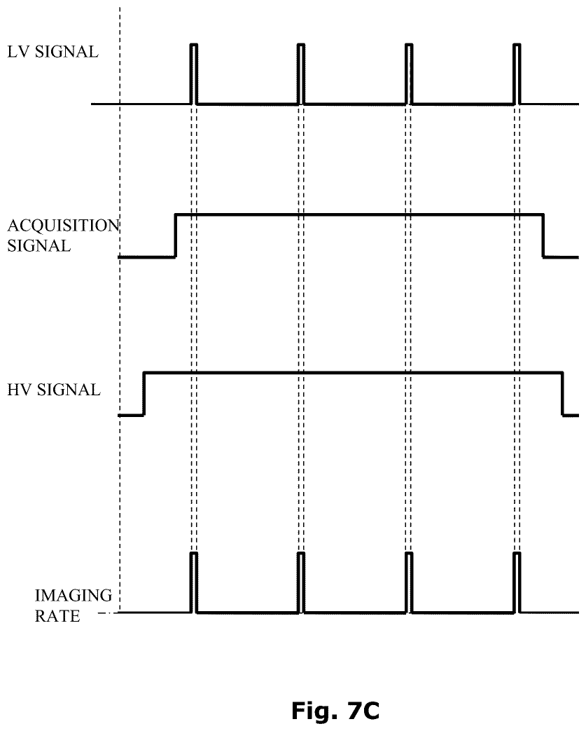

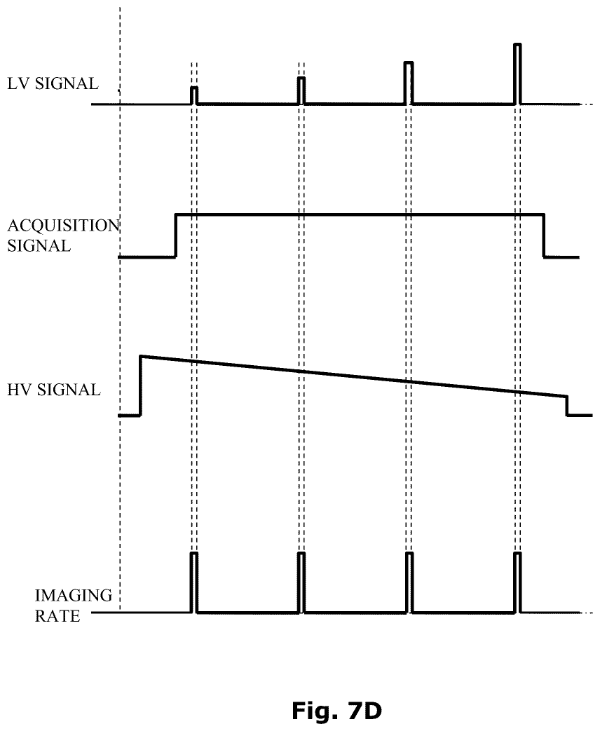

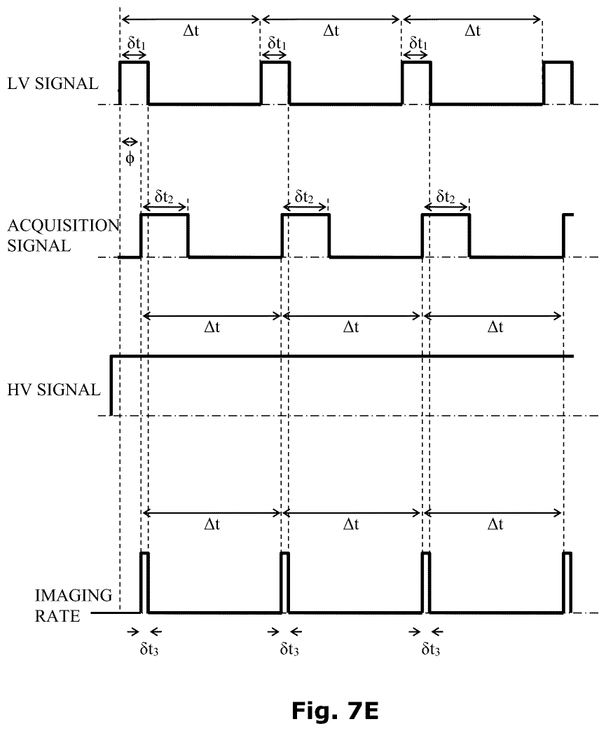

Short, accurate and synchronized gate pulses (at fixed or variable voltage levels=variable mA). The synchronization can be to the sensor/detector/camera “shutter” and/or to a vibrating/rotating examine object. The short pulses yield sharp image (even at high speed movement) and Integration of many synchronized pulses compensate the low energy/brightness of each pulse. See examples of timing diagrams.in FIGS. 7A-E

FIGS. 7A and 7B illustrate how where the duration of a gate pulse is smaller than the duration of the shutter pulse, the effective exposure time may be determined by the duration of the gate pulse regardless of the duration of the High Voltage Acceleration pulse.

FIG. 7C illustrates how a series of LV signal pulses may be used to generate a pulsed imaging rate. It will be appreciated that such a signal may enable an x-ray device to function in a stroboscopic manner.

FIG. 7D illustrates an HV signal having a gradient over time. It is particularly noted that by providing an HV signal having a gradient over time, a number of applications may be possible such as a multispectral device operable to distinguish between materials according to their characteristic x-ray absorption rates.

A multispectral device may be used, for example to identify both soft materials, such as drugs as well as hard materials such as metals. Accordingly, using a multispectral x-ray imager may allow a single device to be used to detect both drugs and weapons for security purposes.

Furthermore, in medical applications, tissue may be differentiated according to their absorption rates. Thus it may be possible to identify rogue bodies such as cancer cells against a background of normal tissue.

In still other applications, the HV signal may be varied to compensate for bodies of varying thickness. So, for example, in a mammogram, the HV signal may be increased and decreased according to the contours of the breast.

FIG. 7D further illustrates how synchronized variation in the low voltage gate signal may compensate for variation in the high voltage acceleration signal such that a constant imaging rate may be maintained,

It is further noted that by the low voltage signal may also be adjusted to compensate for damaged emitters so as to produce a consistent performance of the device over time. Accordingly, any or all of the amplitude, duty cycle and/or frequency or the like may be controlled in order to adjust the LV signal.

Furthermore, self-diagnosis of the x-ray device may be enabled by measuring cathode current, measured between the cathode and the gate electrode, and anode current, measured between the cathode and the anode. Accordingly, electron leakage from the tube may be detected by comparing the measured cathode current and the measured anode current. For example, by monitoring the difference between the measured values or the quotient of the measured values, a leakage index may be calculated indicating the health of the system.

FIGS. 4 and 7E illustrate possible signal profiles of a shutter signal and a gate signal and the resulting imaging rate acquired by an optical imager imaging an irradiated scintillator.

The Activation Signal or Gate Signal is the LV signal triggering the electron emitting construct which has a square profile of comprises a series of gate pulses generated at a regular intervals Δt and having a fixed gate-pulse duration δt1.

The Shutter Signal has the same frequency and consists of a phase shifted series of trigger pulses generated at the same regular intervals Δt and having a fixed shutter-pulse duration δt2.

The Activation Signal is synchronized to the shutter signal such that the start of each shutter-pulse of the shutter signal is offset from the start of each gate-pulse by a phase shift ϕ. Accordingly, the imaging rate is determined by the frequency (same Δt intervals) but the effective exposure time during which the optical imager accumulates optical stimulation is determined by the overlap between the two signals. It is particularly noted that the effective exposure time δt3 may set to be as short as possible regardless of the pulse and/or shutter time.

Various applications of the above described system include using fast and synchronized x-ray pulses for nondestructive stroboscopic industrial radiography tests, for example, inspection of rotating objects and vibration tests.

For example, in airplanes/engines/jet indurates this idea can be used for crack detection in real time in mechanical/rotating loads. Additionally or alternatively accurate examination of rotating objects (the blades) may be possible without removal of their covers using an external x-ray machine.

Accordingly, a method is taught for monitoring periodically moving mechanical components. Such a method includes

The method may further include the high voltage supply establishing an electron accelerating potential between said electron emitting construct and said anode; the controller generating an activation signal comprising a series of gate pulses generated at a regular intervals Δt and having a fixed gate-pulse duration δt1; the shutter controller generating a shutter signal comprising a series of trigger pulses generated at a regular intervals Δt and having a fixed shutter-pulse duration δt2; the synchronizer synchronizing the activation signal with the shutter signal such that the start of each shutter-pulse is offset from the start of a gate-pulse by a phase shift ϕ; and the synchronizer synchronizing the activation signal with the periodically moving mechanical components; sending the activation signal to the digital switching unit.

Accordingly, the method may still further include the digital switch unit activating the low voltage driving circuit to provide the potential difference between the gate electrode and the array of electron sources of the electron emitting construct for the duration of each gate pulse; the electron emitting construct emitting electrons; the high voltage supply accelerating the electrons towards the anode target; and the anode target generating x-rays for the duration of each gate pulse.

Further the x-ray pulses may be directed towards the moving mechanical components; the shutter signal may be sent to the optical imager such that the triggered shutter of the optical imager opens for the duration of each shutter-pulse; and the optical imager accumulates optical stimulation for a duration δt3 equal to the difference between the gate-pulse duration and the phase shift.

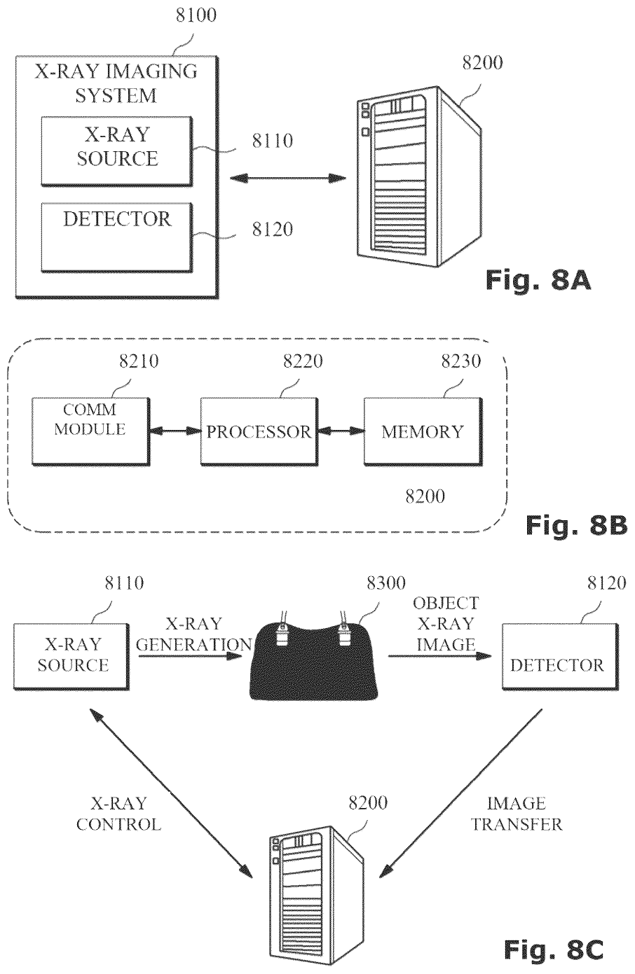

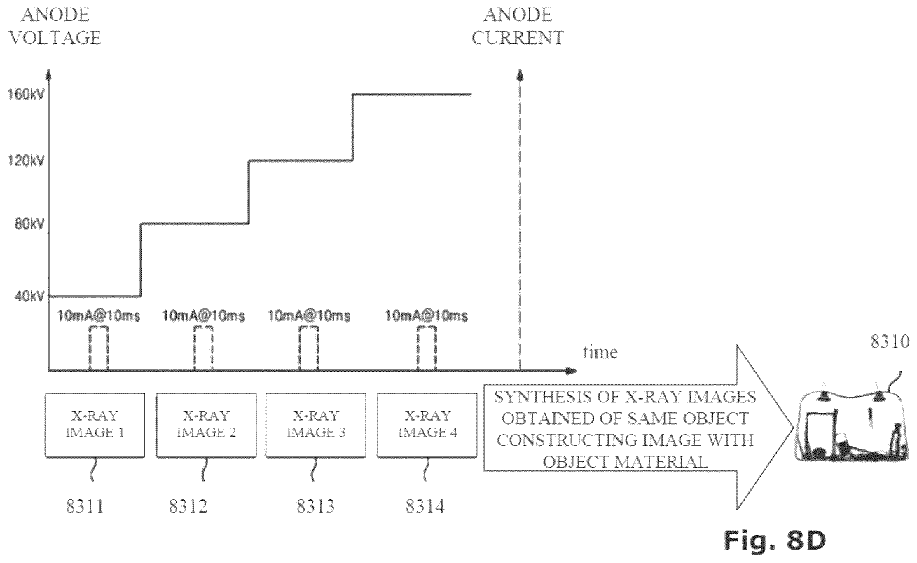

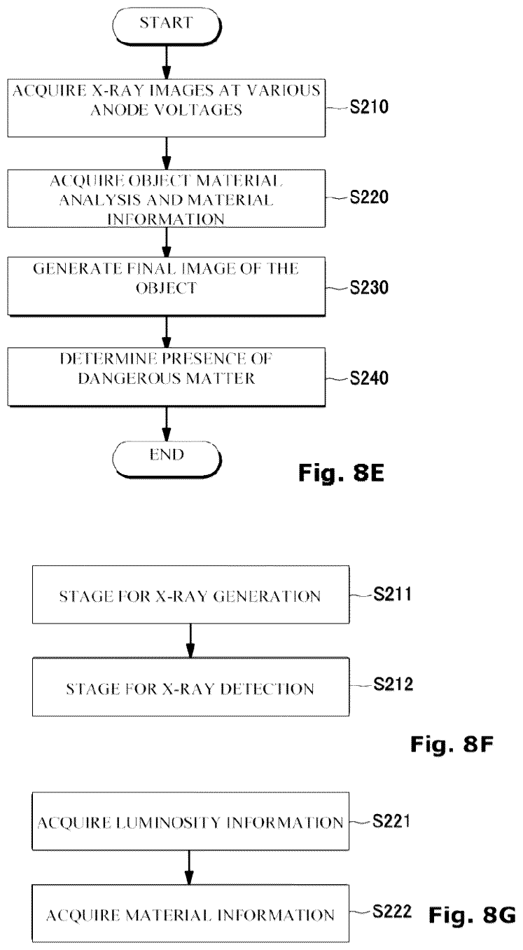

FIG. 8A is a diagram illustrating a configuration of an object material determination system using X-ray imaging according to an embodiment of the present invention, FIG. 8B is a block diagram illustrating the detailed configuration of the server shown in FIG. 8A, FIG. 8C is a diagram illustrating the operation of the object material determination system using X-ray imaging shown in FIG. 8A, and FIG. 8D is a diagram illustrating an experimental example using an object material determination system using X-ray imaging shown in FIG. 8A. Hereinafter, with reference to FIGS. 8A-D, an object material determination system using X-ray imaging according to an embodiment of the present invention (hereinafter, an object material determination system using X-ray imaging) will be described in detail.

It is particularly noted that the material determination system may be used in medical imaging applications to distinguish between the materials of tissues within the body of a subject according to the differences in absorptive properties of various tissues. For example, bone and muscle may be distinguishable as well as organ tissues. In some cases, benign and cancerous growths within the body may be distinctive and distinguishable due to their different absorption of x-rays in a sample set of a plurality of x-ray images obtained by performing x-ray imaging of the object using anode voltages of various sizes.

Referring to FIGS. 8A and 8B, the object material determination system using X-ray imaging is configured to include a control device 8200 that is communicative with the X-ray imaging device 8100 and the X-ray imaging device 8100. The x-ray imaging device 8100 includes an X-ray source 8110 and a detector 8120. The control device 8200 includes a communication module 8210, a memory 8220, and a processor 8230.

The control device 8200 may be implemented as a computing device such as a server or terminal, and may operate in a cloud computing service model such as Software as a Service (Saas), Platform as a Service (PaaS), or Infrastructure as a Service (IaaS). In addition, the control device 200 may be built in a form such as a private cloud, a public cloud, or a hybrid cloud system, The scope of the present invention is not limited thereto. The control device 8200 may perform information transmission and reception with the X-ray imaging device 8100 via a wired or wireless communication network to reconstruct the X-ray image for the subject. The control device 8200 may be separated from the X-ray imaging apparatus 8100 to establish an object material determination system using X-ray imaging on its own. Hereinafter, for convenience of explanation, the control device 8200 is set up to a server for explanation.

Referring to FIG. 8C, briefly describing the operation method of the material determination system of an object using X-ray scanning, the control device 8200 may control the energy of the X-ray generated by the X-ray source 8110 by differently setting the voltage size of the anode having the X-ray source 8110. The X-ray source 8110 may generate X-rays according to the control of the control device 8200 and emit them to the subject 8300. The X-rays that penetrate the subject 8300 are detected by the detector 8120 and the detector 8120 generates an X-ray image of the subject. The generated X-ray image may be transmitted to the control device 8200. The control device 8200 may synthesize a plurality of X-ray images of the subject according to different X-ray energies by the anode voltage size to produce an X-ray image of the final subject. In addition, the material of the objects included in the X-ray image of the subject may be determined based on the contrast difference between the plurality of X-ray images, and related material information may be reflected in the X-ray image of the final subject.

More specifically, the X-ray imaging device 8100 performs X-ray imaging of the subject by the anode voltage size to obtain a plurality of X-ray images of the subject by the anode voltage size. The X-ray imaging device 8100 may be set so that the tube current does not change according to the size of the anode voltage. The X-ray transmittance to the subject may vary when the X-ray energy is changed by varying the size of the anode voltage. For example, the metal is sensitive to the X-ray energy, bodies made of materials such as paper or plastic can be brightly transmitted even when detected using relatively little X-ray energy. Accordingly, if the X-ray image of the subject is detected by changing the X-ray energy generated by setting the anode voltage size differently, the size, thickness, etc. of the material of the subject can be determined.

Such an X-ray imaging device 8100 may include an X-ray source 8110 that generates X-rays for the subject by the anode voltage size by setting the size of the anode voltage differently for each anode voltage size, and a detector 8120 that detects that the X-ray generated by the X-ray source 8110 has penetrated the subject and generates the sample X-ray images by the anode voltage size.

As such, the X-ray imaging device 8100, in which the tube current does not change even if the size of the anode voltage changes, performs X-ray scan of the subject according to the control of the server 8200. X-ray scanning may be performed multiple times by setting the size of the anode voltage differently within a predetermined time. Although not shown in the figure, the X-ray source 8110 may include a bias control unit and a cathode control unit. The X-ray source 8110 may operate at different anode biases and generate unchanged luminance of the X-rays at each bias. The X-ray source 8110 may be in the form of an electric field emitting X-ray source comprising an anode electrode, a cathode electrode, a gate electrode, an electric field emission emitter, and the like in a vacuum tube. The bias control unit of the X-ray source 8110 may be set to the desired voltage between the cathode electrode and the anode electrode. The cathode controller may generate an electric field emission current for the desired time interval during energy scanning and X-ray image acquisition of the subject. The detector 8120 may obtain an X-ray attenuation signal of the object for each X-ray energy step by the anode voltage size.

The server 8200 receives the subject X-ray images by the anode voltage size from the X-ray imaging apparatus 8100, determines the material of the objects included in the subject X-ray images by the anode voltage size based on the difference in the subject X-ray images by the anode voltage size, and synthesizes the subject X-ray images by the anode voltage size and is configured to generate a final X-ray image for the subject by reflecting the material information of the objects included in the anode voltage size subject X-ray images determined in the composite image.

In addition, the server 8200 may be configured to further perform a determination of whether there is a predetermined hazardous material in the final X-ray image based on the material information of the objects included in the sample X-ray images by the anode voltage size. The predetermined hazardous materials may correspond to liquids, gunpowder, and the like, but the scope of the present invention is not limited thereto.

Further, the server 8200 may obtain contrast change information of objects included in the subject X-ray images by anode voltage size based on the contrast difference of the pixels included in each of the sample X-ray images by the anode voltage size. In addition, the server 8200 may further perform to obtain material information of objects included in the object X-ray images by anode voltage size by comparing the contrast change information of the objects included in the object X-ray images by anode voltage size to the standard database in which the contrast change information of the predetermined objects by the anode voltage size is stored. In the standard database, material information for various objects, including hazardous objects such as gunpowder and non-hazardous objects such as clothing, may be mapped and stored with the contrast change information of the X-ray imageed image by anode voltage size. The standard database may be included in the server 8200 or separately constructed outside the server 8200. Thus, the server 8200 may determine the material of the objects included in the subject X-ray images by anode voltage size and obtain related information based on the contrast change information of the objects included in the subject X-ray images by the anode voltage size compared with the contrast change information stored in the standard database.

Referring to FIG. 8B, the server 8200 may itself establish a material determination system of the object using X-ray imaging. The server 8200 may include a communication module 8210 that performs information transmission and reception with the X-ray imaging apparatus 8100. A memory 8220 storing the X-ray program and a processor 8230 that executes the X-ray program may perform the following functions and procedures. The processor 8230 may execute the X-ray program to perform the following functions and procedures.

The processor 8230 receives a sample set of a plurality of X-ray images obtained by performing X-ray imaging of the subject using anode voltages of various sizes obtained by the X-ray imaging device 8100 by performing X-ray scan of the subject at various anode voltage sizes by the X-ray imaging device 8100 through the communication module 8210. Processor 8230 determines the material of objects included in the subject X-ray images by anode voltage size based on differences in subject X-ray images by anode voltage size. Processor 8230 then synthesizes the subject X-ray images by anode voltage size and generates a final X-ray image for the subject reflecting the material information of the objects included in the object X-ray images by the anode voltage size determined in the composite image.

In addition, processor 8230 may execute an X-ray program to determine whether there is a predetermined hazardous substance in the final X-ray image based on the material information of the objects included in the sample X-ray images by anode voltage size.

In addition, the processor 8230 executes an X-ray program to control that the X-ray source 8110 of the X-ray imaging device 8100 generates X-rays for the subject by the anode voltage size that is set differently for a predetermined time, and that the detector 8120 of the X-ray imaging device 8100 detects that the X-ray rays generated by the X-ray source 8110 have penetrated the subject and may control it to generate and transmit the specimen X-ray images by the anode voltage size to the server 8200.

In addition, processor 8230 executes an X-ray program to obtain contrast change information of objects included in the subject X-ray images by anode voltage size based on the contrast difference of the pixels included in each of the subject X-ray images by anode voltage size, and to obtain the material information of the objects included in the object X-ray images by anode voltage size compared to the standard database in which the contrast change information of the objects specified by the anode voltage size is stored. Further, processor 8230 may use deep learning techniques to continuously learn the contrast change information and material information of the X-ray image by anode voltage size for various objects stored in the standard database of objects, the contrast change information and material information of the X-ray image by the anode voltage size for the directly judged subject, and can be used to determine the material of the objects included in the next subject X-ray image.

Referring to FIGS. 8A, 8C and 8D, an experimental example of an object material determination system using X-ray imaging according to an embodiment of the present invention shall be described.

Referring to FIG. 8D, the size of the anode voltage may be set differently to 40 kV, 80 kV, 120 KV, and 160 KV respectively to obtain an X-ray imaging image for the subject 300. Even if the size of the anode voltage changes, the tube current of the X-ray source 8110 may be fixed to 10 mA. The shooting time may be set at 10 ms intervals. The X-ray imaging apparatus 8100 may have an X-ray image 8311 of the subject when the size of the anode voltage is 40 kV, X-ray image 8310 of the subject when the size of the anode voltage is 80 kV, X-ray image 8313 of the subject when the size of the anode voltage is 120 kV, and X-ray imaging image 314 of the subject when the size of the anode voltage is 160 KV can be obtained and transmitted to the server 8200. The server 8200 may synthesize a plurality of X-ray images 8311, 8312, 8313, and 8314 to generate a final X-ray image 8310 for the subject. The final X-ray image 8310 may include material information of objects included in the plurality of X-ray images 8311, 8312, 8313, 8314 generated by the server by comparing with information stored in a standard database based on contrast difference and contrast change information of the plurality of X-ray shooting images 8311, 8312, 8313, 8314. As such, when the subject 8300 is a bag, it is possible to determine whether there is a dangerous substance among the objects present inside the bag.

FIG. 8E is a flowchart illustrating various steps of the method of determining the object material using X-ray imaging according to another embodiment of the present invention, and FIGS. 8F and 8G are diagrams illustrating the detailed steps for some steps of the object material determination method using the X-ray imaging shown in FIG. 8E. The method of determining the object material using X-ray imaging according to the present embodiment is a method using the object material determination system using X-ray imaging described with reference to figures. Each step and detailed process of the object material determination method using X-ray imaging described below may be implemented through the object material determination system using the above-described X-ray imaging. Accordingly, the contents of the embodiments described with reference to FIGS. 8A-D above may be equally applied to the following embodiments, and the contents that overlap with the above-described descriptions shall be omitted from the following.

Referring to FIG. 8E, the object material determination method using X-ray imaging is a material determination method of the object through the communication connection of the X-ray imaging apparatus 8100 and the server 8200, and includes a sample X-ray image acquisition step S210 by anode voltage size, a sample material analysis and information acquisition step S220, and a final X-ray image generation step S230, and may further include a hazardous material presence determination step S240.

The process of obtaining a subject X-ray image by anode voltage size S210 is a step in which the X-ray imaging apparatus 8100 receives a plurality of sample X-ray images by the anode voltage size obtained by performing X-ray imaging of the subject by the anode voltage size, and the server 8200 receives transmission from the X-ray imaging device 8100. The sample material analysis and information acquisition step S220 is a step in which the server 200 determines the material of the objects included in the subject X-ray images by the anode voltage size based on the differences in the subject X-ray images by the anode voltage size. The sample final X-ray image generation step S230 is a step of synthesizing the subject X-ray images by the anode voltage size and generating the final X-ray image for the subject reflecting the material information of the objects included in the object X-ray images by the anode voltage size determined according to step S220 in the composite image. S240) is a step of determining whether a predetermined hazardous substance is present in the final X-ray image based on the material information of the objects contained in the sample X-ray images by the anode voltage size.

Referring to FIG. 8F, the step of obtaining a subject X-ray image by the anode voltage size S210 is a sequence of X-ray generation step S211 where, under the control of the server 8200, generates an X-ray for the subject by the anode voltage size that is set differently for the time set by the X-ray source 8110 of the X-ray imaging device 8100, and detects that the detector 8120 of the X-ray imaging device 8100 detects that the X-ray generated by the X-ray source 8110 has penetrated the subject and generates and transmits the subject X-ray images by the anode voltage size to the server 8200. It may include a step S212.

Referring to FIG. 8G, the sample material analysis and information acquisition step S220 comprises a contrast change information acquisition step (S221) to obtain contrast change information of objects included in the object X-ray images by anode voltage size based on the contrast difference of the pixels included in each of the subject X-ray images by anode voltage size (S221), and the contrast change information of the objects included in the object X-ray images by anode voltage size compared to a standard database containing contrast change information of objects predetermined by anode voltage size. It may include a material information acquisition step S222 to obtain material information of objects.

The method of determining the object material using X-ray imaging described above may also be implemented in the form of a recording medium that includes instructions executable by a computer, such as a program module executed by a computer. The computer-readable medium may be any available medium that can be accessed by a computer, and includes both volatile and non-volatile media, removable and non-removable media. In addition, the computer-readable medium may include a computer storage medium. The computer storage medium may include a computer-readable instruction, It includes both volatile and non-volatile, detachable and non-removable media implemented in any method or technique for storing information such as data structures, program modules or other data.

### Technical Notes

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

1. A method for determining material of an object using an x-ray imaging device in communication with a processor, the method comprising:
the x-ray imaging device capturing a sample set of a plurality of x-ray images obtained by performing x-ray imaging of the object using anode voltages of various sizes;
communicating the sample set to the processor;
the processor receiving the sample set;
the processor comparing the plurality of x-ray images of the sample set to each other;
the processor using differences between the plurality of x-ray images of the sample set to determine material of the object;
the processor synthesizing a composite image of the object from the plurality of x-ray images of the sample set representing different materials constructing the object.

2. The method of claim 1 further comprising:
determining whether there is a hazardous substance in the object based upon material information in the composite image.

3. The method of claim 1 wherein the step of the x-ray imaging device capturing a sample set comprises:
generating x-rays to be directed at the object using anode voltage sizes determined distinctly by a controller;
detecting that x-rays generated by the x-ray source have penetrated the object; and
generating subject x-ray images according to anode voltage size.

4. The method of claim 1 wherein the step of the processor using differences between the plurality of x-ray images of the sample set to determine material of the object comprises:
obtaining contrast change information of features included in the sample set based upon contrast differences of pixels included in each image of the sample set, and
comparing contrast change information of the features included in the sample set to a standard database in which the contrast change information of reference objects is stored.

5. The method of claim 1 wherein the object is a living body and the method further comprising differentiating between biological tissues.

6. The method of claim 5 further comprising detecting cancerous growths in the body.

7. A system for determining material of an object using an x-ray imaging device in communication with a processor, the system comprising:
an x-ray imaging device;
a communication module operable to receive data from the x-ray imaging device and transmit data thereto;
a memory unit for storing x-ray programs; and
a processor operable to execute x-ray program
wherein the x-ray imaging device is operable to receive instructions relating to a plurality of anode voltage sizes.

8. The system of claim 7 wherein a sample set of x-ray images obtained by performing x-ray imaging of the object at various anode voltage sizes is communicated to the processor.

9. The system of claim 7 wherein material composition of the object is determined.

10. The system of claim 8 further comprising a synthesis module operable to synthesize a compound image from the sample set.

11. The system of claim 7 further comprising a material determination module.

12. The system of claim 7 wherein the processor is operable to create a composite x-ray image of the object representing material information of the object.

13. A method for determining material of an object using an x-ray imaging device in communication with a processor, the method comprising:
providing an x-ray emission system comprising:
a field emission type electron emitting construct;
an anode target x-ray source;
a low voltage driving circuit configured to provide a potential difference between a positive terminal wired to a gate electrode and a negative terminal wired to an array of electron sources of the electron emitting construct;
a high voltage supply wired between said electron emitting construct and said anode target x-ray source; and
a control device operable to select potential difference between said electron emitting construct and said anode target x-ray source thereby controlling energy of the x-ray emission from the anode target;
the control device selecting required electron accelerating potential;
the high voltage supply establishing the required electron accelerating potential between said electron emitting construct and said anode target x-ray source;
the electron emitting construct emitting electrons;
the high voltage supply accelerating the electrons towards the anode target; and
the anode target x-ray source emitting x-rays with required energy.

14. The method of claim 13 further comprising:
the control device selecting a series of required electron accelerating potential; and
the high voltage supply producing a corresponding series of electron accelerating potentials between said electron emitting construct and said anode target x-ray source; and
the anode target x-ray source emitting a series of x-ray pulses having energies determined by the corresponding series of electron accelerating potentials.

15. The method of claim 14 further comprising:
directing the series of x-ray pulses at an object;
detecting an x-ray attenuation signal for each of the series of x-ray pulses.

16. The method of claim 15 further comprising generating a series of energy specific x-ray images of the object corresponding to each x-ray energy.

17. The method of claim 16 further comprising synthesizing the series of energy specific x-ray images of the object into a composite image of the object.

18. The method of claim 16 further comprising using differences between the energy specific x-ray images of the sample set to determine material of the object.

19. The method of claim 18 further comprising determining whether there is a hazardous substance in the object based upon material information in the composite image.

20. The method of claim 18 wherein the step of the using differences between the energy specific x-ray images to determine material of the object comprises:
obtaining contrast change information of features included in the series of energy specific x-ray images based upon contrast differences of pixels included in each energy specific x-ray image, and
comparing contrast change information of the features included in the series of energy specific x-ray images to a standard database in which the contrast change information of reference objects is stored.
